(19) United States
(12) Patent Application Publication
(10) Pub. No.: US 2026/0191453 A1
(43) Pub. Date: Jul. 9, 2026

(54) ATRIAL SENSING WITHOUT AN ATRIAL LEAD BY MEDICAL SYSTEMS

(71) Applicant: Medtronic, Inc., Minneapolis, MN (US)

(72) Inventors: Ya-Jian Cheng, Lino Lakes, MN (US); Shantanu Sarkar, Roseville, MN (US); Kevin T. Ousdigian, Shoreview, MN (US)

(21) Appl. No.: 19/125,077

(22) PCT Filed: Oct. 25, 2023
(86) PCT No.: PCT/US2023/035900

§ 371 Date: Apr. 28, 2025

Related U.S. Application Data
(60) Provisional application No. 63/381,536, filed on Oct. 28, 2022.

(51) Int. Cl. A61B 5/29; A61B 5/00; A61B 5/287; A61B 5/327; A61B 5/339; A61B 5/353; A61B 5/361; A61B 5/363

(52) U.S. Cl. CPC A61B 5/29; A61B 5/287; A61B 5/327; A61B 5/339; A61B 5/353; A61B 5/361; A61B 5/363; A61B 5/7267

(57) ABSTRACT

An example system includes processing circuitry configured to apply a trained machine learning model to a cardiac EGM. A training data set of the trained machine learning model includes non-atrial cardiac EGM data and atrial electrical activity labels. The atrial electrical activity labels are determined from atrial cardiac EGM data corresponding to the non-atrial cardiac EGM data. The processing circuitry is configured to, based on the application of the trained machine learning model to the cardiac EGM, identify data points of the cardiac EGM indicative of atrial electrical activity. The processing circuitry is configured to, based on the identification of the data points, determine whether the cardiac EGM satisfies one or more arrhythmia detection criteria and, based on the cardiac EGM satisfying the one or more arrhythmia detection criteria, generate an indication for output, the indication being indicative of the satisfaction of the one or more arrhythmia detection criteria.

This application claims the benefit of U.S. Provisional Patent Application Ser. No. 63/381,536, filed Oct. 28, 2022, the entire content of which is incorporated herein by reference.

### FIELD

The disclosure relates generally to medical systems and, more particularly, medical systems configured to detect cardiac arrhythmia episodes and other cardiac events.

### BACKGROUND

Medical systems have a variety of options with respect to sensors for sensing signals indicative of cardiac activity (e.g., sound activity, electrical activity, and/or the like). In some medical systems, electrodes enable the medical system to capture signals of electrical activity of a heart of the patient. In some examples, one or more implanted leads connected, on one end, to a medical device have on a distal end one or more electrodes positioned adjacent to an inside or outside wall of a cardiac chamber. The medical system may include an implanted lead which may be positioned on an endocardial or epicardial surface of a left ventricle, a right ventricle, a left atrium, or a right atrium. Other example medical devices, such as insertable cardiac monitors (ICMs), may include one or more electrodes coupled to a device housing periphery. Such a device may capture intrinsic electrical signals generated by cardiac muscle and, for example, indicative of depolarizations and repolarizations of the patient's heart, using the electrode(s) coupled to the device housing periphery. Some medical devices may sense electrical activity of the heart via both leads with electrodes and housing electrodes.

The medical system may determine various information from the captured or sensed electrical activity and then, apply that information to affect therapy (e.g., pacing control) for and/or detection of cardiac events. The medical device may generate a cardiac electrogram (EGM) from these captured or sensed signals for monitoring the electrical activity of the patient's heart, for example, to detect one or more types of arrhythmia, such as bradycardia, tachycardia, fibrillation, or asystole (e.g., caused by sinus pause or AV block).

### SUMMARY

Medical devices coupled to intracardiac leads may be considered more invasive and more prone to complications for long-term monitoring of cardiac EGM signals than other options, such as subcutaneously-insertable devices that do not include leads, e.g., ICMs. However, ICMs may be at a disadvantage relative to devices including intracardiac leads for detecting certain cardiac EGM features. For example, it may be more difficult for an ICM to detect atrial electrical activity of the heart, e.g., P-waves, than a device coupled to one or more electrodes in an atrium of the heart. Medical devices that are coupled to one or more leads, but not to an atrial lead, may similarly have difficulty detecting atrial electrical activity of the heart. Consequently, devices without an atrial lead (e.g., ICMs) may oversense some cardiac arrhythmia types and/or undersense other arrhythmia types.

In general, the present disclosure is directed to medical systems, devices, and techniques that facilitate identifying arrhythmias and other cardiac events in a cardiac EGM without utilizing an atrial lead. Instead of capturing atrial electrical activity via one or more atrial leads, the techniques use one or more machine learning models to predict such electrical activity in a non-atrial EGM. In some examples, the training set of data for the machine learning model(s) may include cardiac EGMs collected from non-atrial source, e.g., a ventricular lead, other non-atrial lead, and/or electrodes coupled to a device housing periphery, by a medical system that also included an atrial lead, with the locations of atrial electrical activity in the non-atrial cardiac EGMs labeled based on the contemporaneous atrial electrical activity detections via the atrial lead. In some examples, the training cardiac EGMs may be modified to more closely resemble cardiac EGMs that will be collected by a target medical device, e.g., cardiac EGMs collected by ICMs. In this manner, the techniques of this disclosure may advantageously enable improved accuracy in the identification of true arrhythmias and, consequently, better evaluation of the condition of the patient, for patients having devices without atrial leads.

In some examples, a medical system applying a trained machine learning model to a sensed cardiac EGM may help the identification of a true arrhythmia, even when the sensed cardiac EGM is a non-atrial cardiac EGM (e.g., an EGM that was sensed without using an atrial electrode). Such a medical system may better distinguish between a true arrhythmia and other indications contained in the sensed cardiac EGM than other systems not applying the trained machine learning model or than a clinician attempting to distinguish between true arrhythmia and other indications in a sensed cardiac EGM that was not sensed using an atrial electrode.

In one example, a medical system includes sensing circuitry configured to sense a cardiac electrogram (EGM) of a patient via a plurality of non-atrial electrodes; and processing circuitry configured to: apply a trained machine learning model to the cardiac EGM, the trained machine learning model being previously trained on non-atrial cardiac EGM data and atrial electrical activity labels; based on the application of the trained machine learning model to the cardiac EGM, determine whether at least a portion of the cardiac EGM satisfies one or more arrhythmia detection criteria; and based on the at least a portion of the cardiac EGM satisfying the one or more arrhythmia detection criteria, generate an indication for output, the indication being indicative of the satisfaction of the one or more arrhythmia detection criteria.

In another example, a method includes applying, using processing circuitry of medical system, a trained machine learning model to a cardiac electrogram (EGM), the trained machine learning model being previously trained on non-atrial cardiac EGM data and atrial electrical activity labels; based on the application of the trained machine learning model to the cardiac EGM, determining whether at least a portion of the cardiac EGM satisfies one or more arrhythmia detection criteria; and based on the at least a portion of the cardiac EGM satisfying the one or more arrhythmia detection criteria, generating an indication for output, the indication being indicative of the satisfaction of the one or more arrhythmia detection criteria.

In another example, a non-transitory computer-readable storage medium comprises program instructions that, when executed by processing circuitry of a medical system, cause the processing circuitry to apply a trained machine learning model to the cardiac EGM, wherein a training data set of the trained machine learning model comprises non-atrial cardiac EGM data and atrial electrical activity labels, wherein the atrial electrical activity labels are determined from atrial cardiac EGM data corresponding to the non-atrial cardiac EGM data; based on the application of the trained machine learning model to the cardiac EGM, identify data points of the cardiac EGM indicative of atrial electrical activity; based on the identification of the data points, determine whether the cardiac EGM satisfies one or more arrhythmia detection criteria; and based on the cardiac EGM satisfying the one or more arrhythmia detection criteria, generate an indication for output, the indication being indicative of the satisfaction of the one or more arrhythmia detection criteria.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates the environment of an example medical system in conjunction with a patient.

FIG. 2A is a perspective drawing illustrating an example configuration of the implantable medical device of FIG. 1.

FIG. 2B is a perspective drawing illustrating another example configuration of the implantable medical device of FIG. 1.

FIG. 3 is a block diagram illustrating an example configuration of the IMD of the medical system of FIG. 1.

FIG. 4 is a block diagram illustrating an example configuration of the external device of FIG. 1.

FIG. 5 is a block diagram illustrating an example system that includes a network, external computing devices, such as a server, and one or more other computing devices, which may be coupled to the IMD and external device of FIGS. 1-4.

FIG. 6 is a block diagram illustrating an example configuration of a health monitoring system.

FIG. 7A is a conceptual diagram of an example neural network according to one or more aspects of this disclosure.

FIG. 7B is a conceptual diagram illustrating an example training process for a machine learning model, in accordance with one or more aspects of this disclosure.

FIG. 8 is a conceptual drawing illustrating another example medical device system in conjunction with a patient.

FIG. 9 is a flow diagram illustrating an example operation for training a machine learning model from a corpus of intracardiac EGM segments to predict signal data indicative of atrial electrical activity for a subcutaneous electrocardiogram (ECG).

FIG. 10 is a flow diagram illustrating an example operation for evaluating a cardiac EGM from a leadless medical device by applying a machine learning model to predict signal data indicative of atrial electrical activity of a patient and detecting an arrhythmia based on determining satisfaction of various detection criteria for at least one arrhythmia type.

FIG. 11A and FIG. 11B are each an illustration of a cardiac EGM corresponding to an output configuration of a leadless medical device. The respective illustration of FIG. 11A is a synthesized cardiac EGM that results training a machine learning model from a corpus of intracardiac EGMs from an invasive medical device. The respective illustration of FIG. 11B is a sensed cardiac EGM that results from recording electrical activity of non-atrial tissue and augmenting visual indicators for simulated atrial electrical activity.

Like reference characters denote like elements throughout the description and figures.

### DETAILED DESCRIPTION

A variety of types of devices sense cardiac EGMs. Some devices that sense cardiac EGMs are non-invasive, e.g., using a plurality of electrodes placed in contact with external portions of the patient, such as at various locations on the skin of the patient. The electrodes used to monitor the cardiac EGM in these non-invasive processes may be attached to the patient using an adhesive, strap, belt, or vest, as examples, and electrically coupled to a monitoring device, such as an electrocardiogramalter monitor, or other electronic device. The electrodes are configured to sense electrical signals associated with the electrical activity of the heart or other cardiac tissue of the patient, and, in some examples, to provide these sensed electrical signals to the electronic device for further processing and/or display of the electrical signals. Typically, the non-invasive devices and methods are utilized on a temporary basis, for example to monitor a patient during a clinical visit, such as during a doctor's appointment, or for example for a predetermined period of time, for example for one day (twenty-four hours), or for a period of several days. However, the present disclosure describes techniques for enhancing the non-invasive devices and techniques for enhancing devices with a longer monitoring time (e.g., ICMs). These techniques may result in enhanced ability to detect arrhythmias, enhanced presentation of the cardiac EGM, and/or additional monitoring capabilities, such as enhanced determination of an atrial rate, a ventricular rate, and/or a heart rate variability (HRV) measure.

External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, formerly available from Medtronic plc, of Dublin, Ireland. Such external devices may facilitate relatively longer-term monitoring of patients during normal daily activities and may periodically transmit collected data to a network service, such as the Medtronic Carelink™ Network.

Implantable medical devices (IMDs) also may sense and monitor cardiac EGMs. The IMDs described herein as not including an atrial lead may sense cardiac EGMs via subcutaneous electrodes, cutaneous electrodes, substernal electrodes, extravascular electrodes, intra-muscular electrodes, or any electrodes positioned in (or in contact with) any tissue of a patient except for cardiac tissue in an atrial chamber of a patient's heart. The electrodes used by IMDs to sense cardiac EGMs are typically integrated with a housing of the IMD and/or coupled to the IMD via one or more elongated leads. Example IMDs that monitor cardiac EGMs include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic plc. Some IMDs that do not provide therapy, e.g., implantable patient monitors, sense cardiac EGMs. Examples of such an IMD are the Reveal LINQ™ and LINQ II™ Insertable Cardiac Monitors (ICMs), available from Medtronic plc, which may be inserted subcutaneously. Such IMDs may be less invasive than other IMDs (such as those having an atrial lead), may facilitate relatively longer-term monitoring of patients during normal daily activities, and may periodically transmit collected data to a computing service, such as the Medtronic Carelink™ Network.

Regardless of which of the above IMDs or external devices are used, lacking any atrial sensing capability via electrodes on or within an atrium inhibits the sensing of atrial electrical activity via the cardiac EGMs. In general, sensing via an atrial electrode provides a resolution into certain waveforms (e.g., atrial depolarizations or P-waves) that may be unachievable, or difficult to achieve, from other positions, e.g., subcutaneously. Signals of the atrial electrical activity become diminished/degraded as a distance between the electrode(s) and the patient's heart increases. Therefore, a subcutaneous cardiac EGM in a form of time-stamped electrical activity data is not as accurate as an intracardiac EGM, particularly, with respect to sensing atrial electrical activity. There are a number of other causes/artifacts for inaccurately sensing actual cardiac activity, including a noise signal. Such noise signals may be more prevalent when cutaneous, subcutaneous, or extravascular electrodes are used to sense the cardiac EGM, e.g., due to temporary change in contact between at least one of the electrodes and the tissue where the electrode is located due to relative motion of the electrode and tissue. This and other types of inaccurate sensing may lead to improper analysis of the actual cardiac activity occurring with respect to the patient being monitored. Any false positive indications could lead to incorrect assessment of the patient condition, including provision of therapy and/or sending false alerts to medical personnel responsible for the care of the patient being monitored. As such, the techniques of this disclosure may improve the accuracy of a sensed EGM and/or any analysis based thereon.

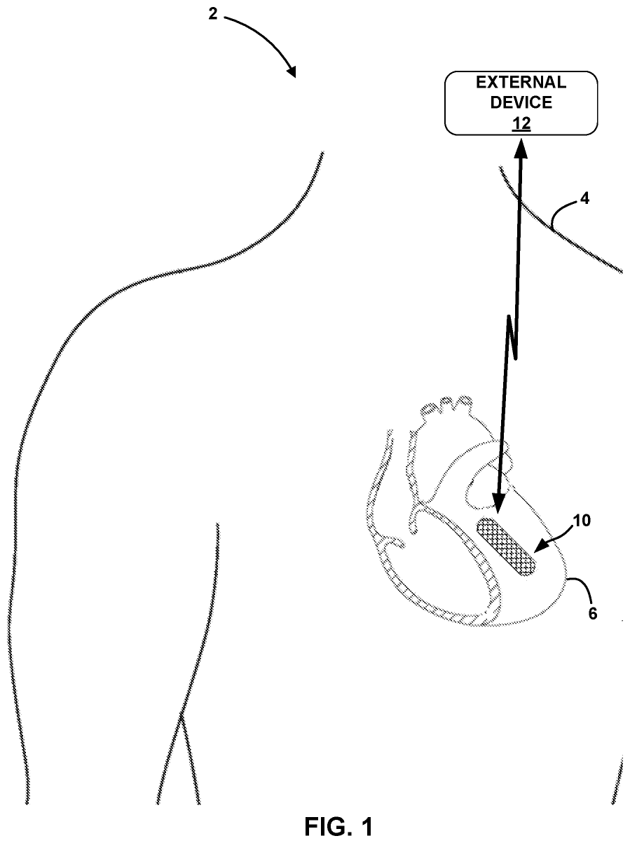

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral/thoracic location illustrated in FIG. 1). IMD 10 may be positioned near the sternum near or just below the level of heart 6 of patient 4, e.g., at least partially within the cardiac silhouette. IMD 10 includes a plurality of electrodes (not shown in FIG. 1) and is configured to sense a cardiac EGM via the plurality of electrodes. In some examples, IMD 10 takes the form of the Reveal LINQ™ or LINQ II™ ICM.

External device 12 may be a computing device with a display viewable by the user and an interface for providing input to external device 12 (e.g., a user input mechanism). In some examples, external device 12 may be a notebook computer, tablet computer, workstation, one or more servers, cellular phone, personal digital assistant, or another computing device that may run an application that enables the computing device to interact with IMD 10.

External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

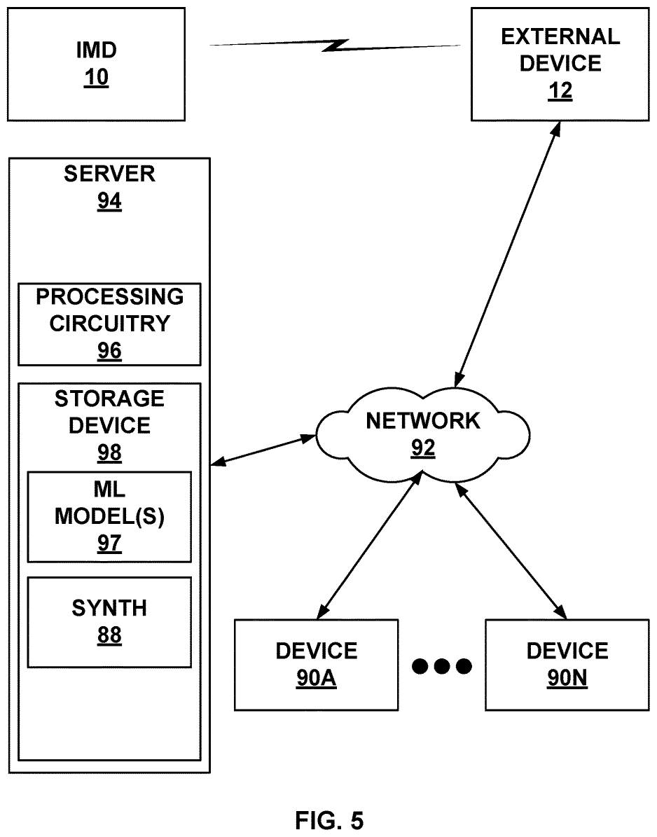

External device 12 may be used to configure operational settings for IMD 10. External device 12 may be used to retrieve data from IMD 10. The retrieved data may include values of physiological parameters measured by IMD 10, indications of episodes of arrhythmia or other maladies detected by IMD 10, and physiological signals recorded by IMD 10. For example, external device 12 may retrieve cardiac EGM segments recorded by IMD 10, for example, due to IMD 10 determining that an episode of asystole or another malady occurred during the segment. As will be discussed in greater detail below with respect to FIG. 5, one or more remote computing devices may interact with IMD 10 in a manner similar to external device 12, e.g., to program IMD 10 and/or retrieve data from IMD 10, via a network.

Processing circuitry of medical system 2, e.g., of IMD 10, external device 12, and/or of one or more other computing devices not shown in FIG. 1, may be configured to perform the example techniques for applying one or more trained machine learning models to non-atrial cardiac EGM data to identify atrial electrical activity within the non-atrial cardiac EGM. In some examples, the processing circuitry of medical system 2 analyzes the cardiac EGM to determine whether one or more of a plurality of arrhythmia detection criteria are satisfied. Each of the detection criteria may be configured to detect one or more indicators of an occurrence of an arrhythmia type in the cardiac EGM. For example, the detection criteria may be used to detect an occurrence of bradycardia, atrial and/or ventricular tachycardia, atrial and/or ventricular fibrillation, and/or asystole Although described in the context of examples in which IMD 10 that senses the cardiac EGM comprises an ICM, example systems including one or more implantable or external devices of any type configured to sense a cardiac EGM may be configured to implement the techniques of this disclosure. In some examples, processing circuitry of medical system 2 may analyze the cardiac EGM to determine at least one of an atrial rate, a ventricular rate, or an HRV measure.

The present disclosure describes medical systems, devices, and techniques enabling identification, enhancement, or substitution (e.g., replacement or recreation) of atrial electrical activity data when performing cardiac monitoring operation(s). Instead of an atrial lead and an electrode positioned in the atrial chamber for capturing and recording atrial signals as time-stamped atrial electrical activity data, the present disclosure describes techniques utilizing one or more machine learning models for predicting or identifying the occurrence of the atrial signals that would have been captured via lead(s), and then applying that prediction/identification towards performing the cardiac monitoring operation(s).

The present disclosure further describes devices, such as IMD 10, without an atrial lead that implement the techniques of this disclosure and as a result, may be able to achieve a same or similar accuracy as if an atrial lead was present. IMD 10 may use the techniques to detect atrial electrical activity with accuracy approaching sensing via atrial electrodes and leads and then, incorporate the detected atrial electrical activity into time-stamped electrical activity of the patient. The medical systems, devices, and techniques described herein train one or more machine learning models using training data generated by devices with an actual atrial lead. IMD 10 represents an example of medical devices that are either leadless or, otherwise, lacking electrodes on or within an atrium.

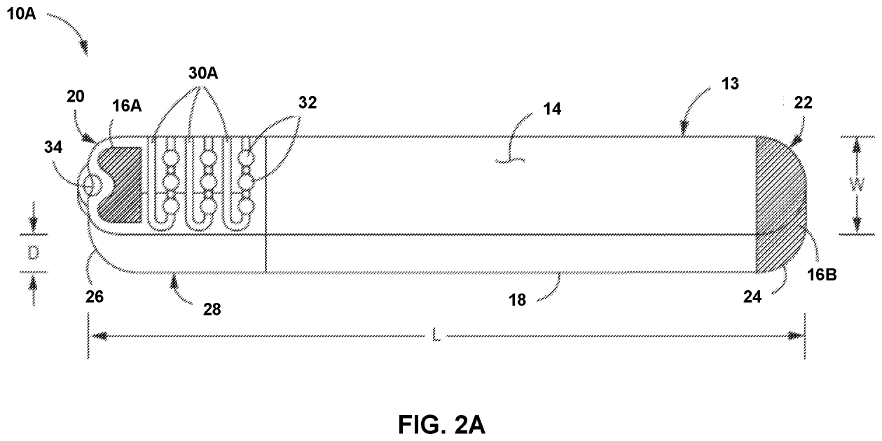

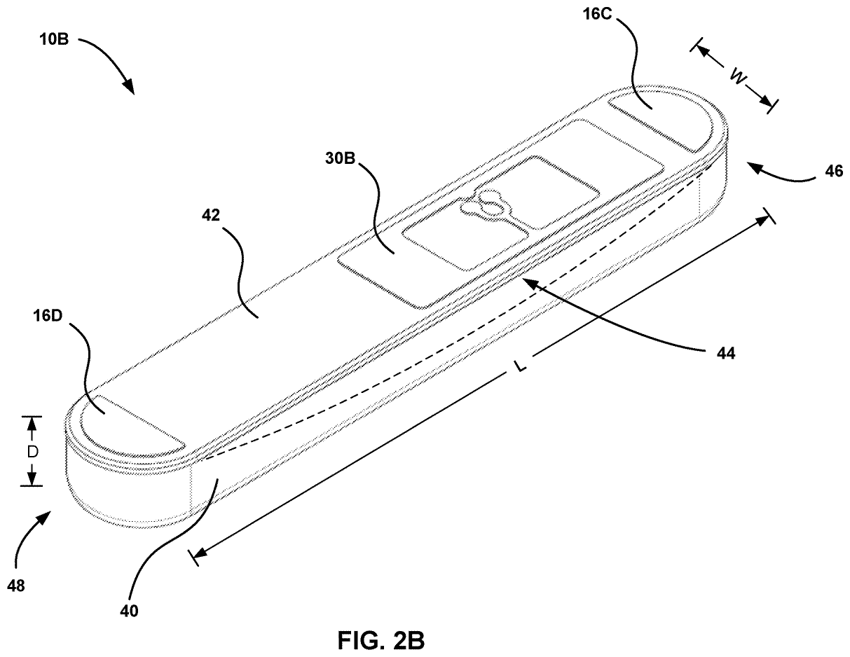

FIG. 2A is a conceptual drawing illustrating an IMD 10A, which may be an example configuration of IMD 10 of FIG. 1 as an ICM. In the example shown in FIG. 2A, may be implemented as a monitoring device having housing 13, proximal electrode 16A and distal electrode 16B. Housing 13 may further comprise first major surface 14, second major surface 18, proximal end 20, and distal end 22. Housing 13 encloses electronic circuitry located inside the IMD 10A and protects the circuitry contained therein from body fluids. Electrical feedthroughs provide electrical connection of electrodes 16A and 16B.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, the spacing between proximal electrode 16A and distal electrode 16B may range from 30 millimeters (mm) to 55 mm, 35 mm to 55 mm, and from 40 mm to 55 mm and may be any range or individual spacing from 25 mm to 60 mm. In addition, IMD 10A may have a length L that ranges from 30 mm to about 70 mm. In other examples, the length L may range from 40 mm to 60 mm, 45 mm to 60 mm and may be any length or range of lengths between about 30 mm and about 70 mm. In addition, the width W of major surface 14 may range from 3 mm to 10 mm and may be any single or range of widths between 3 mm and 10 mm. The thickness of depth D of IMD 10A may range from 2 mm to 9 mm. In other examples, the depth D of IMD 10A may range from 2 mm to 5 mm and may be any single or range of depths from 2 mm to 9 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within the patient, the first major surface 14 faces outward, toward the skin of the patient while the second major surface 18 is located opposite the first major surface 14. In addition, in the example shown in FIG. 2A, proximal end 20 and distal end 22 are rounded to reduce discomfort and irritation to surrounding tissue once inserted under the skin of the patient. IMD 10A, including instrument and method for inserting IMD 10 is described, for example, in U.S. Pat. No. 11,311,312, issued on Apr. 26, 2022, which is herein incorporated by reference in its entirety.

Proximal electrode 16A and distal electrode 16B are used to sense cardiac signals, e.g., EGM signals, intra-thoracically or extra-thoracically, which may be sub-muscularly or subcutaneously. EGM signals may be stored in a memory of IMD 10A, and data may be transmitted via integrated antenna 30A to another medical device, which may be another implantable device or an external device, such as external device 12. In some example, electrodes 16A and 16B may additionally or alternatively be used for sensing any bio-potential signal of interest, which may be, for example, an EGM, EEG, EGM or a nerve signal, from any implanted location.

In the example shown in FIG. 2A, proximal electrode 16A is in close proximity to the proximal end 20 and distal electrode 16B is in close proximity to distal end 22. In this example, distal electrode 16B is not limited to a flattened, outward facing surface, but may extend from first major surface 14 around rounded edges 24 and/or end surface 26 and onto the second major surface 18 so that the electrode 16B has a three-dimensional curved configuration. In some examples, electrode 16B is an uninsulated portion of a metallic, e.g., titanium, part of housing 13.

In the example shown in FIG. 2A, proximal electrode 16A is located on first major surface 14 and is substantially flat, and outward facing. However, in other examples proximal electrode 16A may utilize the three-dimensional curved configuration of distal electrode 16B, providing a three-dimensional proximal electrode (not shown in this example). Similarly, in other examples distal electrode 16B may utilize a substantially flat, outward facing electrode located on first major surface 14 similar to that shown with respect to proximal electrode 16A.

The various electrode configurations allow for configurations in which proximal electrode 16A and distal electrode 16B are located on both first major surface 14 and second major surface 18. In other configurations, such as that shown in FIG. 2A, only one of proximal electrode 16A and distal electrode 16B is located on both major surfaces 14 and 18, and in still other configurations both proximal electrode 16A and distal electrode 16B are located on one of the first major surface 14 or the second major surface 18 (e.g., proximal electrode 16A located on first major surface 14 while distal electrode 16B is located on second major surface 18). In another example, IMD 10A may include electrodes on both major surface 14 and 18 at or near the proximal and distal ends of the device, such that a total of four electrodes are included on IMD 10A. Electrodes 16A and 16B may be formed of a plurality of different types of biocompatible conductive material, e.g., stainless steel, titanium, platinum, iridium, or alloys thereof, and may utilize one or more coatings such as titanium nitride or fractal titanium nitride. It should be noted that, in the example of FIG. 2A, IMD 10A does not include an atrial electrode.

In the example shown in FIG. 2A, proximal end 20 includes a header assembly 28 that includes one or more of proximal electrode 16A, integrated antenna 30A, anti-migration projections 32, and/or suture hole 34. Integrated antenna 30A is located on the same major surface (e.g., first major surface 14) as proximal electrode 16A and is also included as part of header assembly 28. Integrated antenna 30A allows IMD 10A to transmit and/or receive data. In other examples, integrated antenna 30A may be formed on the opposite major surface as proximal electrode 16A, or may be incorporated within the housing 13 of IMD 10A. In the example shown in FIG. 2A, anti-migration projections 32 are located adjacent to integrated antenna 30A and protrude away from first major surface 14 to prevent longitudinal movement of the device. In the example shown in FIG. 2A, anti-migration projections 32 include a plurality (e.g., nine) small bumps or protrusions extending away from first major surface 14. As discussed above, in other examples anti-migration projections 32 may be located on the opposite major surface as proximal electrode 16A and/or integrated antenna 30A. In addition, in the example shown in FIG. 2A, header assembly 28 includes suture hole 34, which provides another means of securing IMD 10A to the patient to prevent movement following insertion. In the example shown, suture hole 34 is located adjacent to proximal electrode 16A. In one example, header assembly 28 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1. IMD 10B of FIG. 2B may be configured substantially similarly to IMD 10A of FIG. 2A, with differences between them discussed herein.

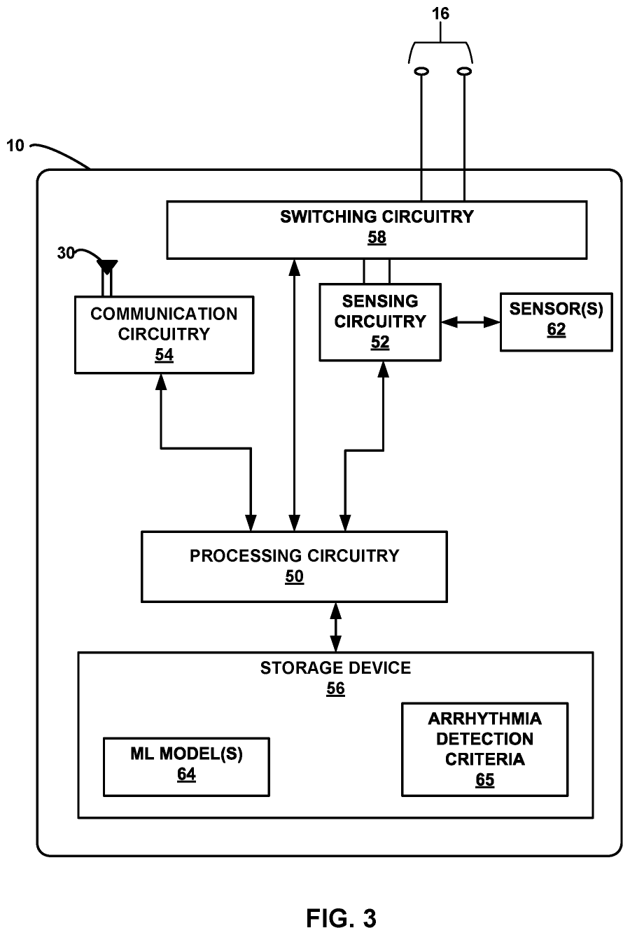

IMD 10B may include a leadless, subcutaneously-implantable monitoring device, e.g. an ICM. IMD 10B includes housing having a base 40 and an insulative cover 42. Proximal electrode 16C and distal electrode 16D may be formed or placed on an outer surface of cover 42. Various circuitries and components of IMD 10B, e.g., described below with respect to FIG. 3, may be formed or placed on an inner surface of cover 42, or within base 40. In some examples, a battery or other power source of IMD 10B may be included within base 40. In the illustrated example, antenna 30B is formed or placed on the outer surface of cover 42, but may be formed or placed on the inner surface in some examples. In some examples, insulative cover 42 may be positioned over an open base 40 such that base 40 and cover 42 enclose the circuitries and other components and protect them from fluids such as body fluids.

Circuitries and components may be formed on the inner side of insulative cover 42, such as by using flip-chip technology. Insulative cover 42 may be flipped onto a base 40. When flipped and placed onto base 40, the components of IMD 10B formed on the inner side of insulative cover 42 may be positioned in a gap 44 defined by base 40. Electrodes 16C and 16D and antenna 30B may be electrically connected to circuitry formed on the inner side of insulative cover 42 through one or more vias (not shown) formed through insulative cover 42. Insulative cover 42 may be formed of sapphire (i.e., corundum), glass, parylene, and/or any other suitable insulating material. Base 40 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between electrodes 16C and 16D may range from 30 millimeters (mm) to 50 mm, from 35 mm to 45 mm, or be approximately 40 mm. In addition, IMD 10B may have a length L that ranges from 30 mm to about 70 mm. In other examples, the length L may range from 40 mm to 60 mm, 45 mm to 55 mm, or be approximately 45 mm. In addition, the width W may range from 3 mm to 10 mm, such as approximately 8 mm. The thickness of depth D of IMD 10B may range from 2 mm to 9 mm, from 3 to 5 mm, or be approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within the patient, outer surface of cover 42 faces outward, toward the skin of the patient. In addition, as shown in FIG. 2B, proximal end 46 and distal end 48 are rounded to reduce discomfort and irritation to surrounding tissue once inserted under the skin of the patient. In addition, edges of IMD 10B may be rounded.

FIG. 3 is a block diagram illustrating an example configuration of IMD 10 of FIG. 1 in accordance with one or more techniques described herein. In the illustrated example, IMD 10 includes electrodes 16, which may correspond to electrodes 16A and 16B of FIG. 2A and/or electrodes 16C and 16D of FIG. 2B, an antenna 30, which may correspond to antenna 30A of FIG. 2A and/or antenna 30B of FIG. 2B, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

One example of IMD 10 is the Reveal LINQ™ or LINQ II™ ICM, available from Medtronic plc, which may be considered relatively non-invasive or least-invasive amongst IMDs. IMD 10 may be inserted subcutaneously and extravascularly to record cardiac electrical activity of patient 4. IMD 10 may store time-stamped cardiac EGM data, e.g., electrocardiogram (ECG or EKG) data. As described in greater detail below, IMD 10 may be configured to implement one or more trained machine learning (ML) model(s) 64 operative to enhance IMD 10, for example, with the ability of IMD 10 to identify atrial electrical activity within cardiac EGMs (e.g., extra-cardiac EGMs, far-field EGMs, subcutaneous EGMs, or the lick), based on insight learned from a corpus of intracardiac EGM data provided by other devices, such as pacemakers and/or ICDs. As described herein, a remote computing system uses such a corpus as a basis of truth for training machine learning model(s) 64 to accurately sense electrical activity of a heart.

IMD 10 may be configured to, when implanted in patient 4, to monitor EGMs of patient 4, including cardiac EGMs, continuously (which may be continually (e.g., ceaseless), periodically at a periodicity that may be predetermined, such as on the order of a number of minutes, a number of seconds, a number of milliseconds or the like, or based on events that may occur) and uninterrupted over periods of time and without human intervention. By continuously, monitoring EGM data of patient 4, IMD 10 may overcome limitations of a clinician who cannot be continuously with the patient over the time that IMD 10 may be monitoring the EGMs of patient 4. Furthermore, IMD 10 may process such data in a complex manner that a clinician may be unable to perform. For example, a clinician may be unable to mentally examine non-atrial cardiac EGM data and determine what in that non-atrial cardiac EGM data may be indicative of atrial activity. Furthermore, even if a clinician where somehow able to determine atrial activity in the non-atrial cardiac EGM data, the clinician, when considering the non-atrial cardiac EGM data may be unable to determine which non-atrial cardiac EGM data may be indicative of true arrhythmia or a type of arrhythmia that patient 4 may be experiencing.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be implemented as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense electrical activity from a position within patient 4, as controlled by processing circuitry 50. Sensing circuitry 52 may sense signals from electrodes 16, e.g., to record electrical activity of a heart and produce a subcutaneous cardiac EGM as a memorialization of that electrical activity. In some examples, sensing circuitry 52 may include analog-to-digital conversion circuitry to digitize the cardiac EGM, e.g., for analysis by processing circuitry 50 and/or storage in storage device 56. In order to facilitate patient health monitoring operations (e.g., arrhythmia detection) processing circuitry 50 may be configured to apply cardiac EGM data to one or more machine learning model(s) 64 configured to output values indicating, e.g., the probability, that data points on the EGM are indicative of atrial electrical activity, e.g., P-waves. For example, the one or more machine learning model(s) 64 may output an identification of data points of the cardiac EGM data that are indicative of atrial electrical activity.

Sensing circuitry 52 and/or processing circuitry 50 may be configured to detect cardiac depolarizations (e.g., P-waves or R-waves), such as when the cardiac EGM amplitude crosses a sensing threshold, matches a waveform pattern, and/or satisfies another criterion. As described herein, conventional medical devices often rely on sensing atrial electrical activity for accurate indicia of waveforms, for instance, to the benefit of the above detection method for cardiac depolarizations.

Processing circuitry 50 may detect an asystole episode based on determining that the cardiac electrogram satisfies an asystole detection criterion. The asystole detection criterion may be absence of a cardiac depolarization for a threshold period of time. In such examples, processing circuitry 50 may determine that the cardiac EGM satisfies the asystole detection criterion based on reaching a predetermined time interval from detection of a cardiac depolarization without receiving another cardiac depolarization indication from sensing circuitry 52.

Sensing circuitry 52 may also provide one or more digitized cardiac EGM signals to processing circuitry 50 for analysis, e.g., for use in cardiac rhythm discrimination, and/or for analysis to determine whether one or more arrhythmia detection criteria are satisfied according to the techniques of this disclosure. In some examples, based on satisfaction of the asystole detection criterion, processing circuitry 50 may store a segment of the digitized cardiac EGM corresponding to the suspected asystole as episode data in storage device 56. The digitized cardiac EGM segment may include samples of the cardiac EGM spanning the period of time for which sensing circuitry 52 did not indicate detection of a depolarization, as well as a period of time before and/or after this period of time during which depolarizations were detected. Processing circuitry 50 of IMD 10, and/or processing circuitry of another device that retrieves the episode data from IMD 10, may analyze the cardiac EGM segment to determine whether one or more false asystole detection criteria are satisfied according to the techniques of this disclosure. For example, processing circuitry 50 may determine a suspected asystole (or other arrhythmia) by comparing the digitized cardiac EGM signals, or information derived therefrom, to arrhythmia detection criteria 65. Arrhythmia detection criteria 65 may include arrhythmia detection criteria that are known to those skilled in the art. Arrythmia detection criteria 65 may, for example, include a time between consecutive P-waves meeting a threshold (e.g., being less than the threshold, or being less than or equal to the threshold).

Sensing circuitry 52 may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 30. In addition, processing circuitry 50 may communicate with a networked computing device via an external device (e.g., external device 12) and a computer network, such as the Medtronic CareLink® Network. Antenna 30 and communication circuitry 54 may be configured to transmit and/or receive signals via inductive coupling, electromagnetic coupling, Near Field Communication (NFC), Radio Frequency (RF) communication, Bluetooth®, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include episode data for suspected arrhythmias and/or indications that suspected arrhythmias satisfied one or more false asystole detection criteria.

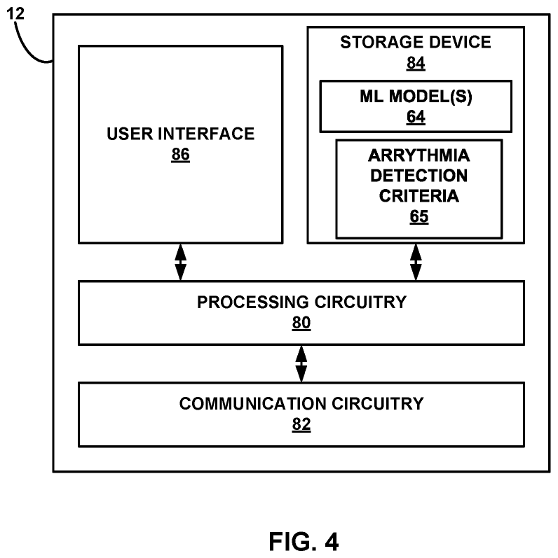

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC, RF communication, Bluetooth®, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution. Storage device 84 may include machine learning model(s) 64 and/or arrhythmia detection criteria 65.

Data exchanged between external device 12 and IMD 10 may include operational parameters and/or configuration settings. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., asystole episode data) to external device 12. In turn, external device 12 may receive the collected data from IMD 10 and store the collected data in storage device 84.

Processing circuitry 80 may implement any of the techniques described herein to process cardiac EGMs received from IMD 10, such as applying machine learning model(s) 64 to EGMs to predict atrial electrical activity, determining whether an EGM and associated predicted atrial electrical activity is indicative of arrhythmia (e.g., through the use of arrhythmia detection criteria 65), or the like. For example, processing circuitry 80 may confirm or reject initial detections (e.g., by IMD 10) of arrhythmias by way of determining satisfaction of true and/or false detection criteria and/or may enhance visualization of a cardiac EGM segment (for example, by amplifying or inserting a P-wave into the cardiac EGM segment). For example, processing circuitry 50 may use the cardiac EGM segment, or data associated therewith, for determining a heart rate, HRV, atrial rate, ventricular rate, and detecting arrhythmias, such as tachyarrhythmias and asystole.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., cardiac EGMs, indications of detections of arrhythmia episodes, indications of cardiac depolarizations, and/or other information. In addition, user interface 86 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 5 is a block diagram illustrating an example system that includes a network 92, external computing devices, such as a server 94, and one or more other devices 90A-90N (collectively, “devices 90”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point via a second wireless connection

An access point (not shown) may include a device that connects other devices to network 92 via any of a variety of connections, such as telephone dial-up, digital subscriber line (DSL), or cable modem connections. In other examples, the access point may be coupled to network 92 through different forms of connections, including wired or wireless connections. In some examples, the access point may be a user device, such as a tablet or smartphone, that may be co-located with the patient. IMD 10 may be configured to transmit data, such as cardiac EGM data and indications that one or more asystole detection criteria or false asystole detection criteria are satisfied, to access point. The access point may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via devices 90 or their personal/work computing devices. One or more aspects of the illustrated system of FIG. 5 may be implemented with general network technology and functionality, which may be similar to that provided by the Medtronic CareLink® Network.

In some examples, one or more of devices 90 provide the intracardiac EGM data for the training corpus described herein. For example, one or more of devices 90 may be IMDs having atrial leads from which the training corpus is obtained. One or more of implanted devices 90 may collect raw signal data (e.g., EGM data) from each implanted (e.g., atrial) lead (and, in some examples, from electrodes not located on an implanted lead) and transmit processed information (e.g., cardiac EGMs with waveform indicia and/or parameters) to server 94 or external device 12 for training machine learning model(s) 64 to predict atrial electrical activity from non-atrial sensed data and/or to predict other cardiac information, such as atrial rate, ventricular rate, and/or an HRV measure.

IMDs amongst devices 90 may be implanted subcutaneously or submuscularly on the left/right midaxillary of patient 4 or other subcutaneous locations on patient 4 such as at a pectoral location or abdominal location. An example IMD 90A includes an extravascular implantable cardioverter-defibrillator (EV-ICD). Such a device provides the intracardiac EGM data to help build an example corpus. In some pacemakers, a lead can be employed to locate a bipolar electrode pair in a heart chamber to provide an additional near field EGM sense signal from which the P-wave or R-wave can be sensed (depending on the location of the bipolar electrode pair) and through which pacing pulses can be applied to the atrium or ventricle.

Cardiac EGMs sensed via extravascular electrodes may include noise, e.g., due to changing contact with tissue and/or orientation relative to heart, in a similar manner as described herein with respect to subcutaneous electrodes.

An example IMD 90A includes an implantable loop recorder (ILR) or insertable cardiac monitor (ICM) that is a subcutaneous, single-lead, electrocardiographic (ECG). The MEDTRONIC® Reveal™ insertable loop recorder is a form of implantable cardiac monitor that is intended to be implanted subcutaneously and has a pair of sense electrodes spaced apart on the device housing that are used to pick up the cardiac far field EGM which in this case is also characterized as a “subcutaneous ECG”. The Reveal™ insertable loop recorder samples and records one or more segments (depending on the programmed operating mode) of such far field EGM or subcutaneous ECG signals when the patient feels the effects of an arrhythmic episode and activates the recording function by applying a magnet over the site of implantation. Without such a lead, the above ILR becomes leadless and may no longer benefit from a same accuracy.

Subcutaneous EGMs are used for sensing in subcutaneous ICMs, pacemakers, and/or ICDs, monitoring arrhythmias in ILRs, and as the leadless ECG diagnostic in pacemakers and ICDS. Simulated simultaneous recordings of subcutaneous EGMs and surface ECG signals from electrodes placed directly over the subcutaneous locations have similar amplitude and signal to noise ratio. For at least this reason, the corpus can be used to build the machine learning model to map subcutaneous EGM segments to specific waveforms.

However, sensing the subcutaneous EGM has fundamental limitations in comparison with sensing closely spaced endocardial EGMs: lower signal to noise ratio, postural variation, and no direct access to atrial EGMs. By deploying a fully trained machine learning model to predict data points indicative of certain waveforms of atrial electrical activity, the computing service operating, for example, on server 94, may achieve a same or similar accuracy through non-invasive means when compared to ICDs and other devices with a transvenous lead.

In some examples, processing circuitry 96 of server 94 executes synthesizer 88 which may utilize EGM signal data sensed by devices having an atrial lead. Synthesizer 88 may alter sensed EGM data (e.g., pre-process sensed EGM data) from such devices to resemble non-atrial cardiac sensed EGM data from IMD 10 or an external sensing device (e.g., a watch, a patch, a necklace, or the like). For example, synthesizer 88 may superimpose EGM waveforms from more than one set of electrodes and/or from different leads, may time-delay (e.g., positively or negatively) one or more EGM waveforms, may transform (e.g., non-linear filter, non-linear phase, etc.) one or more sections of or one or more EGM waveforms, and/or may add or suppress noise of one or more sections of or one or more EGM waveforms. Synthesizer 88 may alter the sensed EGM data to become more like the EGM data sensed by a target device, such as IMD 10 or an external device, such as a patch, a watch, a necklace, or the like. Such an alteration may be achieved using expert selected parameters or by the use of a machine learning model, such as a Generative Adversarial Network (GAN). For example, a GAN may be an unsupervised model that may automatically discover patterns in input data and generate output based on the patterns. The GAN may include a generator and a classifier. To train the GAN, real data and generated data may be input to the classifier which may attempt to classify the data as real or generated. The GAN may learn from the classifications in an attempt to generate a higher percentage of generated output that the classifier classifies as real data. The real data that may be used as input to the GAN may include intracardiac EGM data, non-atrial sensed cardiac EGM data from device(s) having an atrial lead, information indicative of atrial electrical activity (e.g., an atrial sensed P-wave), and/or sensed cardiac EGM data from devices not having an atrial lead. Through the use of the GAN, processing circuitry 96 may determine a manner of synthesizing the non-atrial cardiac EGM data.

The synthesized non-atrial cardiac sensed EGM data, and information indicative of atrial electrical activity (e.g., an atrial sensed P-wave) may be input to train machine learning model(s) 97 such that a trained machine learning model(s) 97 may predict atrial electrical activity (e.g., an atrial P-wave) based on non-atrial cardiac sensed EGM data, such as that sensed by IMD 10. While the use of synthesizer 88 to preprocess or alter sensed EGM data from devices having an arterial lead, in some examples, such as examples where sensed EGM data from various electrodes not of an arterial lead resemble EGM data from a target device (e.g., IMD 10), synthesizer 88 may be bypassed and the sensed EGM data may be input to train machine learning model(s) 97 without preprocessing.

In some examples, processing circuitry 96 may determine QRS subtracted EGM data, for example, using template matching with a QRS template, using adaptive filtering in a ventricular intracardiac EGM, or using a principal component analysis (PCA) (e.g., of machine learning model(s) 97) to separate out the QRS complexes from other cardiac EGM data (e.g., intracardiac EGM data, non-atrial cardiac EGM data, and/or synthesized non-atrial cardiac data. Processing circuitry 96 may use the QRS subtracted EGM data, the intracardiac EGM data, the non-atrial cardiac EGM data, and/or the synthesized non-atrial cardiac EGM data for training machine learning model(s) 97. In some examples, the QRS subtracted EGM data may be input into a parallel neural network, different from a neural network for other EGM data and processing circuitry 96 may combine the two neural networks using an ensemble network.

Machine learning model(s) 97 may be example(s) of trained machine learning model(s) 64, but in any state of training (e.g., not trained, in the process of training, trained, etc.). As such processing circuitry 96 may train machine learning model(s) 97 and after training machine learning model(s) 97 may transmit trained machine learning model(s) 64 to IMD 10 and/or external device 12. In this manner, IMD 10 and/or external device 12 may obtain trained machine learning model(s) 64.

In some examples, one or more of devices 90 include a tablet computer, a smart phone, or other smart device, located with a clinician, by which the clinician may program, receive alerts from, and/or interrogate IMD 10. For example, the clinician may access data collected by IMD 10 through device 90N, such as when patient 4 is in in between clinician visits, to check on a status of a medical condition. In some examples, the clinician may enter instructions for a medical intervention for patient 4 into an application executed by device 90N, such as based on a status of a patient condition determined by IMD 10, external device 12, server 94, or any combination thereof, or based on other patient data known to the clinician.

In further examples, IMD 10 and/or external device 12 may generate and/or output an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 94 includes a storage device 98, e.g., to store data retrieved from IMD 10, and processing circuitry 96. Although not illustrated in FIG. 5 computing devices 90 may similarly include a storage device and processing circuitry. Processing circuitry 96 may include one or more processors that are configured to implement functionality and/or process instructions for execution within server 94. For example, processing circuitry 96 may be capable of processing instructions stored in storage device 98. Processing circuitry 96 may include, for example, microprocessors, DSPs, ASICs, FPGAs, or equivalent discrete or integrated logic circuitry, or a combination of any of the foregoing devices or circuitry. Accordingly, processing circuitry 96 may include any suitable structure, whether in hardware, software, firmware, or any combination thereof, to perform the functions ascribed herein to processing circuitry 96. Processing circuitry 96 of server 94 and/or the processing circuity of computing devices 90 may implement any of the techniques described herein to analyze cardiac EGMs received from IMD 10, e.g., to determine whether asystole and false asystole criteria are satisfied.

Storage device 98 may include a computer-readable storage medium or computer-readable storage device. In some examples, storage device 98 includes one or more of a short-term memory or a long-term memory. Storage device 98 may include, for example, RAM, DRAM, SRAM, magnetic discs, optical discs, flash memories, or forms of EPROM or EEPROM. In some examples, storage device 98 is used to store data indicative of instructions for execution by processing circuitry 96.

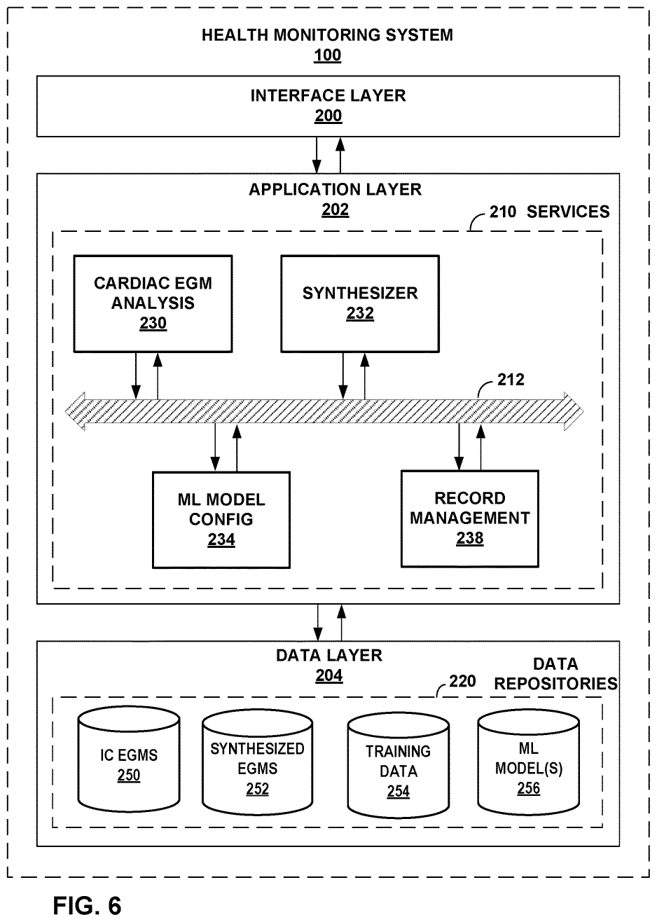

FIG. 6 is a block diagram illustrating an example configuration of a health monitoring system. While described herein as an example of server 94 of FIG. 5, health monitoring system 100 may additionally, or alternatively, be an example system 2 (FIG. 1), or of any one or more components of FIG. 5.

Health monitoring system 100 may include an interface layer 200, an application layer 202, and a data layer 204. Interface layer 200 may include communication circuitry by which health monitoring system 100 may communicate with other devices, such as IMD 10, external device 12, and/or devices 90 via network 92. Interface layer 200 include circuitry similar to communication circuitry 54 of FIG. 3 and/or communication circuitry 82 of FIG. 4. In some examples, interface layer 200 may also include a user interface for receiving information from and/or providing information to, a user. Such a user interface may be similar to user interface 86 of FIG. 4.

Application layer 202 may include a plurality of services 210. Services 210 may include cardiac EGM analysis 230, synthesizer 232, machine learning model configurer 234, and record management 238. Such services may be services running on, or executable by, processing circuitry 96. Such services may be interconnected by bus 212. Data layer 204 may include a plurality of data repositories 220. Data repositories 220 may include intracardiac EGMs 250, synthesized EGMs 252, training data 254, and machine learning model(s) 256.

Cardiac EGM analysis 230 may be configured to analyze a cardiac EGM, such as intracardiac EGMs 250. For example, cardiac EGM analysis 230 may analyze a potential training cardiac EGM to determine whether synthesis is desirable, to determine whether a potential training cardiac EGM should be included in training data 254, etc. For example, if the potential training cardiac EGM is not associated with a device having an atrial lead or is not an intracardiac EGM or associated with an intracardiac EGM, such a potential training cardiac EGM may not be desired and, in some examples, be omitted from training data 254. if the potential training cardiac EGM is not associated with a device having an atrial lead or is not an intracardiac EGM or associated with an intracardiac EGM, such a potential training cardiac EGM may not be desired and, in some examples, be omitted from training data 254.

Synthesizer 232 may be an example of synthesizer 88 of FIG. 5 and may operate accordingly. For example, synthesizer 232 may synthesize any of intracardiac EGMs 250 to generate synthesized EGMs 252. Machine learning model configurer 234 may configure machine learning model(s) 256 by training machine learning model(s) 256 using training data 254. Record management 238 may control the storage of data within data repositories 220.

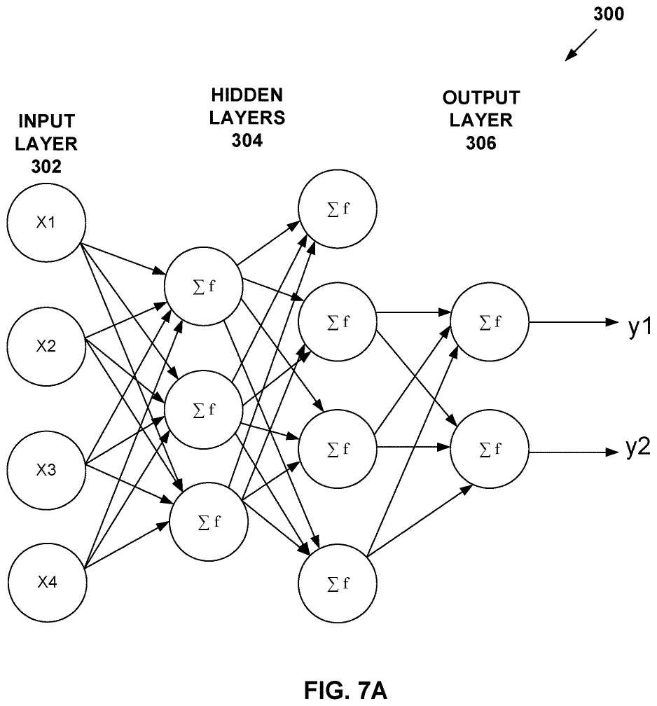

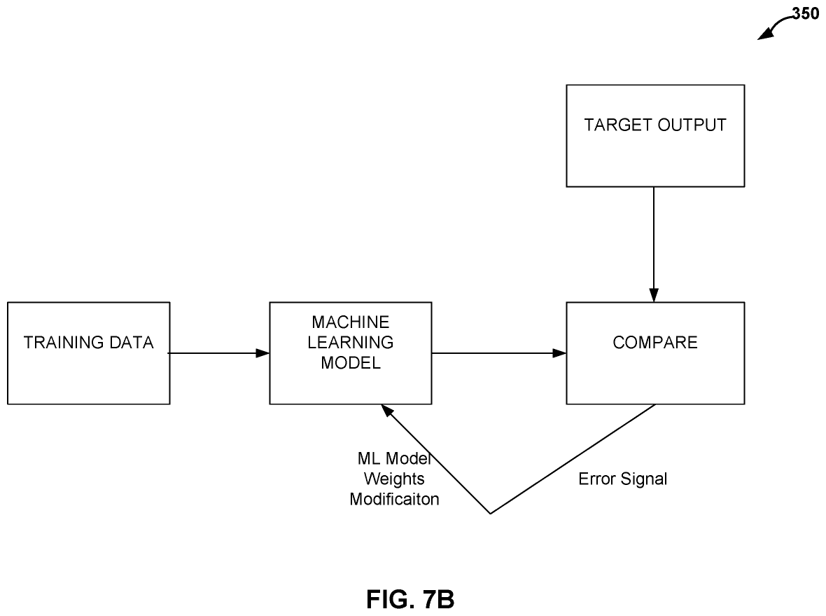

FIG. 7A is a conceptual diagram of an example neural network according to one or more aspects of this disclosure. While discussed with reference to processing circuitry 50 of IMD 10, machine learning algorithm(s) 64 may, additionally, or alternatively, be executed by processing circuitry of other devices, such as processing circuitry 80 of external device 12 or processing circuitry 96 of server 94.

Neural network 300 may be an example of trained machine learning model(s) 64. Neural network 300 may include input layer 302, hidden layers 304, and output layer 306. For example, input layer 302 may obtain inputs x₁-x₄. While shown with four inputs, it should be understood that input layer 302 may obtain any number of inputs. Examples of possible inputs to input layer 302 include sensed EGM signals sensed by IMD 10. Input layer 302 may extract features and/or values, for example, data points, from the inputs. For example, input layer 302 may extract features and/or values from sensed EGM signals of IMD 10.

Hidden layers 304 may process input data, such as inputs x₁-x₄. For example, processing circuitry 50 applies hidden layers 304 the extracted features and/or values to generate a prediction of atrial electrical activity, such as a P-wave, corresponding to information (e.g., one or more data points) in the sensed EGM signals from IMD 10, or other predictions, such as an atrial rate, a ventricular rate, a HRV or the like. For example, each circle within hidden layers 304 may represent a generation of a prediction of atrial electrical activity based on one or more data points in the sensed EGM signals from IMD 10. In some examples, each circle within hidden layers 304 may generate a prediction of atrial electrical activity based on different one or more data points, or overlapping one or more data points, than each other circle within hidden layers 304.

Output layer 306 may further process data from hidden layers 304 and generate output data, such as y₁ and y₂, which may include a prediction of atrial electrical activity based on the predictions of hidden layers 304. While shown with two outputs, it should be understood that output layer 306 may generate any number of outputs. Example possible outputs of output layer 306 may include a prediction of a P-wave, characteristics or morphology of the P-wave (e.g., location, amplitude, area under the curve, slope, etc.), atrial rate, ventricular rate, HRV, or the like.

For example, processing circuitry 50 may classify probabilities determined by machine learning model(s) 64 or apply a function, such as a softmax function. In some examples, such classification or function may be part of machine learning model(s) 64. to generate output probabilities. For example, the classification or function may be used to generate an overall prediction of atrial electrical activity coinciding with a given segment of a non-atrial EGM segment. Processing circuitry 50 may use this prediction of the P-wave, or characteristics or morphology thereof, to determine whether the non-atrial EGM segment is indicative of arrhythmia.

In some examples, processing circuitry 50 or processing circuitry 80 may use output of trained machine learning model(s) 64 to predict where atrial depolarizations are in a non-atrial cardiac EGM and output indications of such predicted atrial depolarizations on a user interface, such as user interface 86, for clinicians view, so that the clinicians may better classify cardiac EGMs (e.g., non-atrial cardiac EGMS) stored by IMD 10 as being indicative of arrhythmia or not, and/or of what type of arrhythmia. For example, processing circuitry 50 or processing circuitry 80 may apply a marker or line on a displayed EGM indicating where the predicted atrial activity is in the displayed EGM. In some examples, processing circuitry 50 and/or processing circuitry 80 may further include an interval plot of predicted A-A intervals along with the sensed V-V intervals.

FIG. 7B is a conceptual diagram illustrating an example training process for a machine learning model, in accordance with one or more aspects of this disclosure. Process 350 may be used to train machine learning model(s) 97. A machine learning model may be implemented using any number of models for supervised and/or reinforcement learning, such as but not limited to, an artificial neural network, a decision tree, naïve Bayes network, support vector machine, or k-nearest neighbor model, to name only a few examples. In some examples, one or more of IMD 10, external device 12, server 94, and/or computing device(s) 90 initially trains the machine learning model based on a corpus of training date. Such training data may include, for example, intracardiac EGM data, non-atrial cardiac EGM data, synthesized non-atrial EGM data, QRS subtracted EGM data, atrial electrical activity labels, and/or the like. For example, when server 94 trains machine learning model(s) 97, server 94 may transmit trained machine learning model(s) (e.g., machine learning model(s) 64) to external device 12 and/or IMD 10 for use in predicting or identifying an arrhythmia in non-atrial cardiac EGM data.

While training machine learning model(s) 97, processing circuitry 96 may compare a prediction or classification by machine learning model(s) 97 with a target output, and an error signal and/or machine learning model modification weights may sent/applied to the machine learning model(s) 97 based on the comparison to modify/update machine learning model(s) 97. For example, one or more of IMD 10, external device 12, server 94, and/or computing device(s) 90 may, for each training instance in the training set, modify, based on the training data, machine learning model(s) 97 and/or machine learning model(s) 64 change the one or more arrhythmia detection criteria which may be used to predict or identify an arrhythmia in non-atrial cardiac EGM data.

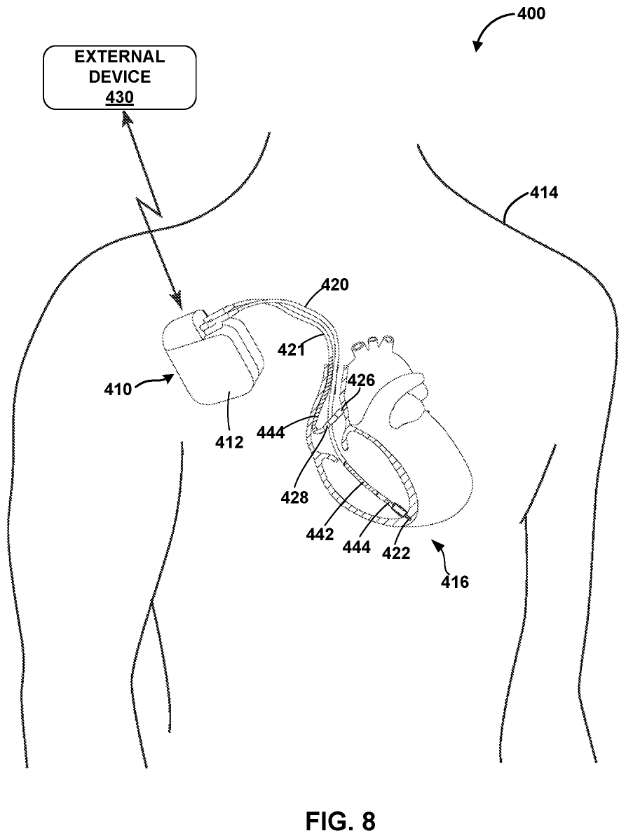

FIG. 8 is a conceptual drawing illustrating an example medical device system 400 in conjunction with a patient 414. Medical device system 400 is an example of a medical device system having an IMD with an atrial lead. In the illustrated example, medical device system 400 includes an IMD 410 coupled to a ventricular lead 420 and an atrial lead 421. IMD 410 may be an ICD capable of delivering pacing, cardioversion and defibrillation therapy to the heart 416 of a patient 414, and will be referred to as ICD 410 hereafter. Ventricular lead 420 and atrial lead 421 are electrically coupled to ICD 410 and extend into the patient's heart 416. Ventricular lead 420 includes electrodes 422 and 424 shown positioned on the lead in the patient's right ventricle (RV) for sensing ventricular EGM signals and pacing in the RV. Atrial lead 421 includes electrodes 426 and 428 positioned on the lead in the patient's right atrium (RA) for sensing atrial EGM signals and pacing in the RA.

Ventricular lead 420 additionally carries a high voltage coil electrode 442, and atrial lead 421 carries a high voltage coil electrode 444, used to deliver cardioversion and defibrillation shocks. The term “anti-tachyarrhythmia shock” may be used herein to refer to both cardioversion shocks and defibrillation shocks. In other examples, ventricular lead 420 may carry both of high voltage coil electrodes 442 and 444, or may carry a high voltage coil electrode in addition to those illustrated in the example of FIG. 1. IMD 10A may use both ventricular lead 420 and atrial lead 421 to acquire cardiac electrogram (EGM) signals from patient 414 and to deliver therapy in response to the acquired data. Medical device system 400 is shown as having a dual chamber ICD configuration, but other examples may include one or more additional leads, such as a coronary sinus lead extending into the right atrium, through the coronary sinus and into a cardiac vein to position electrodes along the left ventricle (LV) for sensing LV EGM signals and delivering pacing pulses to the LV. In other examples, a medical device system may be a single chamber system, or otherwise not include ventricular lead 420.

Processing circuitry, sensing circuitry, and other circuitry configured for performing the techniques described herein are housed within a sealed housing 412. Housing 412 (or a portion thereof) may be conductive so as to serve as an electrode for pacing or sensing or as an active electrode during defibrillation. As such, housing 412 is also referred to herein as “housing electrode” 412.

ICD 410 may transmit EGM signal data and cardiac rhythm episode data acquired by ICD 410, as well as data regarding delivery of therapy by ICD 410, to an external device 430. External device 430 may be a computing device that may be used in a home, ambulatory setting, clinic, or hospital setting, to communicate with ICD 410 via wireless telemetry and may function similarly to external device 12 of FIGS. 1, 4 or 5, or server 94 of FIG. 5. External device 430 may be coupled to, or be part of, a remote patient monitoring system, such as Carelink®, available from Medtronic plc, of Dublin, Ireland. External device 430 may be, as examples, a programmer, external monitor, a server, or consumer device, e.g., smart phone. In the example where ICD 410 is one of external devices 90 (FIG. 5), EGM signal data sensed by ICD 410 may be used as input to synthesizer 95 and/or machine learning model(s) 64, for training machine learning model(s) 64. For example, synthesizer 95 may utilize EGM signal data sensed by ICD 410 to synthesize non-atrial cardiac sensed EGM data to resemble non-atrial cardiac sensed EGM data from IMD 10 or an external sensing device (e.g., a watch, a patch, a necklace, or the like). The synthesized non-atrial cardiac sensed EGM data, and information indicative of atrial electrical activity (e.g., an atrial sensed P-wave) may be input to train machine learning model(s) 64 such that a trained machine learning model(s) 64 may predict atrial electrical activity based on non-atrial cardiac sensed EGM data, such as that sensed by IMD 10.

External device 430 (which may operate similarly to external device 12 of FIGS. 1, 4, and 5) may be used to program commands or operating parameters into ICD 410 for controlling its functioning, e.g., when configured as a programmer for ICD 410. External device 430 may be used to interrogate ICD 410 to retrieve data, including device operational data as well as physiological data accumulated in IMD memory. The interrogation may be automatic, e.g., according to a schedule, or in response to a remote or local user command. Programmers, external monitors, and consumer devices are examples of external devices 430 that may be used to interrogate ICD 410. Examples of communication techniques used by ICD 410 and external device 430 include radiofrequency (RF) telemetry, which may be an RF link established via Bluetooth, WiFi, or medical implant communication service (MICS).

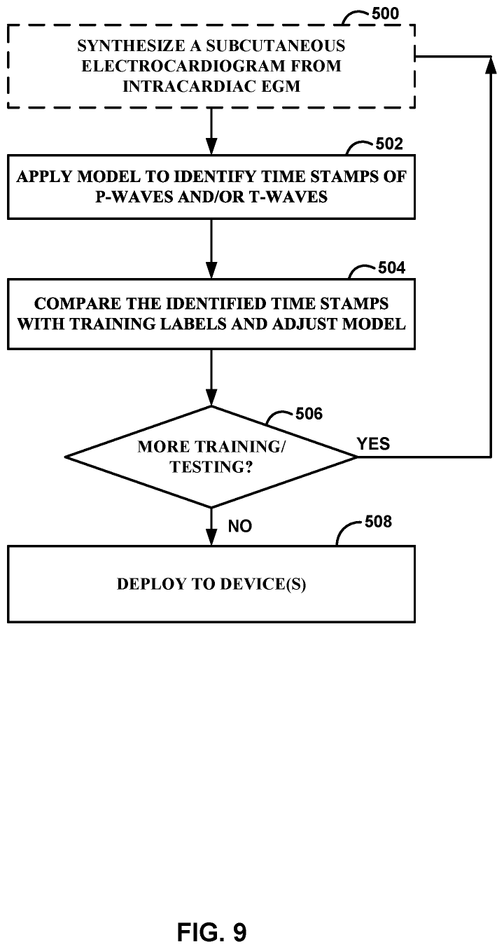

FIG. 9 is a flow diagram illustrating an example operation for training a machine learning model from a corpus of intracardiac EGM segments to predict signal data indicative of atrial electrical activity for a patient having a medical device that is incapable of sensing intracardiac EGM signals or not programmed to sense intracardiac EGM signals. Such a medical device may be configured to perform only non-invasive (or relatively non-invasive) monitoring operations, for example, by configuring (e.g., manufacturing) the medical device to be leadless or employ a lead for (at most) subcutaneously placing an electrode in the patient's body. A computing service may use a corpus of intracardiac EGM data to train one or more machine learning model(s) to identify data points in a subcutaneous EGM segment corresponding to an atrial signal (e.g., an atrial EGM). In some examples, the subcutaneous EGM segment may have been previously sensed by sensing circuitry 52 of IMD 10.

In some examples, processing circuitry (e.g., processing circuitry 96 of server 94 (FIG. 5), or processing circuitry of a computing service) may synthesizes subcutaneous EGM from an intracardiac EGM (500). It should be noted that, in some examples, synthesizing the subcutaneous EGM may not be performed, such as when EGM data from a device having an atrial lead, resembles subcutaneous EGM data. The synthesized subcutaneous EGM may be similar to EGM data from medical devices that are incapable of capturing an intracardiac EGM from an atrial heart chamber (e.g., atrial EGM). Processing circuitry 96 may use a corpus of intracardiac EGMs to identify appropriate signal data to correlate with a subcutaneous EGM and generate the synthesized subcutaneous EGM. For example, the signal data may correspond to electrical activity of a heart chamber (e.g., atrial chamber or ventricular chamber), submuscular electrical activity, and/or the like.

In an example where ICD devices provide their recorded intracardiac EGMs and waveform data for the corpus of intracardiac EGMs, processing circuitry 96 may can use this corpus as a source of truth for interpreting signals of cardiac activity for medical devices without an atrial lead.

One technique for synthesizing a subcutaneous EGM from the corpus of intracardiac EGMs is to superimpose the ICD waveforms of multiple ICD leads (and/or multiple sets of electrodes) on a sample from the corpus. Such a techniques may first build templates defining morphology and pattern attributes of the ICD waveforms of multiple ICD leads and then, use those templates in superimposing, for example, by modifying signal data of the sample. In addition, or alternatively, processing circuitry 96 may modify the sample intracardiac EGM by adding a negative or positive time-delay to one or more of the ICD waveforms. In another technique, processing circuitry 96 performs a transformation such as a linear, non-linear filtered, and/or non-linear phased transformation of one or more of the ICD waveforms of multiple ICD leads (and/or multiple sets of electrodes). Another technique may add or suppress noise and/or artifacts to the ICD waveforms of multiple ICD leads (and/or multiple sets of electrodes) before superimposing these waveforms to generate the synthesized subcutaneous EGM. Parameters for determining when and/or how much to modify the sample intracardiac EGM may be pre-determined and/or dynamically selected through the machine learning model (e.g., Generative Adversarial Network (GAN)).

Processing circuitry 96 may apply machine learning model(s) 97 to identify time stamps of certain waveforms in the synthesized EGM or the otherwise input EGM, such as P-waves and/or T-waves (502). These identified time stamps may include x-axis coordinates on the synthesized EGM or otherwise input EGM corresponding to y-axis coordinates of the waveforms, which are data points presenting amplitude values.

Processing circuitry 96 may compare the identified time stamps with training labels from the original sample intracardiac EGM and adjust the model (504). ICDs include one or more transvenous leads, including at least one lead in an atrial chamber, and therefore, the signal data recorded by these leads include waveforms corresponding to electrical activity from the atrial chamber. The same signal data can be used to identify data points indicative of the atrial electrical activity in the signal data (e.g., waveforms) recorded by medical devices that do not have a transvenous lead. The identified data points may not include the same signal data (e.g., waveforms) but may refer to locations where the same atrial electrical activity most likely will occur given that the synthesized EGM is a modification of the original sample intracardiac EGM. Therefore, the training labels refer to the above waveforms and their observed time stamps in the sample intracardiac EGM, which form a basis of truth for the P-waves and/or T-waves in the synthesized EGM configured to resemble waveforms recorded by the medical devices without any transvenous lead positioned near atrial tissue or in EGMs otherwise input to train machine learning model(s) 97. There may be training labels for arrhythmia detection criterion parameter values predicted by machine learning model based on the synthesized EGM or otherwise input EGM. Atrial/Ventricle rate and/or HRV are examples of these parameters.

Processing circuitry 96 may proceed to determine whether more training/testing is to be performed on machine learning model(s) 97 (506). There are a number of metrics and criteria to use in determining whether the model is to be further trained/tested. For example, a proper determination can be made based on a criterion that is set to one or more accuracy thresholds.

Based on determining that machine learning model(s) 97 are to be further trained/tested (the “YES” path from block 506), the example operation of FIG. 6 repeats a training operation for a next sample intracardiac EGM from the corpus from ICD devices. Based on determining that machine learning model(s) 97 require no further training/testing (the “NO” path from block 506), the example operation of FIG. 6 proceeds to deploy machine learning model(s) 97 to subscribed medical devices and/or external devices (e.g., IMD 10 and/or external device 12) and then, ends (140). In some examples, rather than determine whether the model requires no further training, processing circuitry 96 may determine whether machine learning model(s) 97 are sufficiently trained for deployment. If machine learning model(s) 97 are sufficiently trained for deployment, processing circuitry 96 may deploy machine learning model(s) 97 to subscribed medical devices and/or external devices (e.g., IMD 10 and/or external device 12) and then, rather than ending, continue to repeat a training operation for a next sample intracardiac EGM from the corpus from ICD devices (e.g., returning to box 100). In this manner, machine learning model(s) 97 may be continuously updated. In some examples, rather than deploy machine learning model(s) 97 after each update, processing circuitry 96 may deploy updated machine learning model(s) 97 on a periodic basis or upon demand, for example, by external device 12.

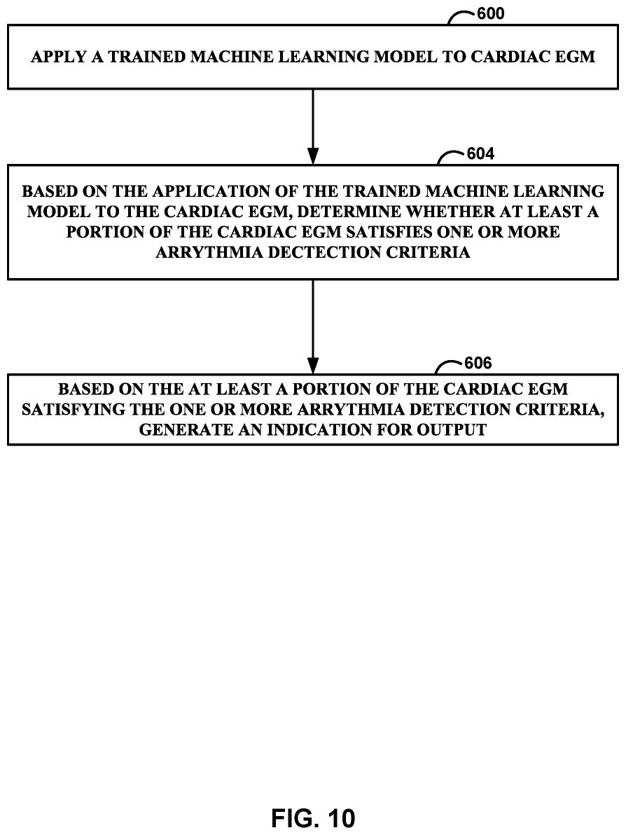

FIG. 10 is a flow diagram illustrating an example operation for evaluating a cardiac EGM from a leadless medical device by applying a machine learning model to predict signal data indicative of atrial electrical activity of a patient and detecting an arrhythmia based on determining satisfaction of various detection criteria for at least one arrhythmia type. While discussed with respect to processing circuitry 50 of IMD 10, it should be noted that the techniques of the example of FIG. 9 may be implemented by processing circuitry 50 of IMD 10, processing circuitry 80 of external device 12, processing circuitry 96 of server 94, processing circuitry of another computing service, or any combination thereof.

Processing circuitry 50 may apply a trained machine learning model to the cardiac EGM (600). For example, processing circuitry 50 may apply trained machine learning model(s) 64 to a cardiac EGM sensed by IMD 10. For example, the trained machine learning model may be previously trained on non-atrial cardiac EGM data and atrial electrical activity labels. For example, a training data set of trained machine learning model(s) may include non-atrial cardiac EGM data (e.g., synthesized EGM data) and atrial electrical activity labels, wherein the atrial electrical activity labels are determined from atrial cardiac EGM data (e.g., intracardiac EGM data). The atrial electrical activity labels may correspond to atrial activity in the non-atrial cardiac EGM data.

Based on the application of the trained machine learning model to the cardiac EGM, processing circuitry 50 may determine whether at least a portion of the cardiac EGM satisfies one or more arrhythmia detection criteria (604). For example, processing circuitry 50 may identify data points of the cardiac EGM indicative of atrial electrical activity. For example, processing circuitry 50 executing trained machine learning model(s) 64, may identify data points of the cardiac EGM (sensed by IMD 10) that are predicted to be associated with atrial electrical activity.

Based on the identification of the data points, processing circuitry 50 For example, processing circuitry 50 may apply one or more arrhythmia detection criteria to the cardiac EGM data including the identified data points. In some examples, these criteria may include criteria for discriminating between at least some actual arrhythmias and falsely detected arrhythmias. Such arrhythmia detection criteria may be known to those skilled in the art and/or may be arrhythmia detection criteria developed in the future.

Based on the cardiac EGM satisfying the one or more arrhythmia detection criteria, processing circuitry 50 may generate an indication for output (606). For example, processing circuitry 50 may generate an indication that is indicative of the satisfaction of the one or more arrhythmia detection criteria. For example, the indication may include an alert, an alarm, or other notification that the one or more arrhythmia detection criteria have been met. In some examples, the indication may include the sensed EGM signal or portion thereof, that was determined to satisfy the one or more arrhythmia detection criteria. In some examples, the indication may include patient parameters, such as atrial rate, ventricular rate, HRV, or the like. In some examples, processing circuitry 50 may enhance a sensed EGM signal to insert or modify a P-wave (and/or T-wave) in the sensed EGM data for enhanced visibility purposes. In some examples, processing circuitry 50 may output the indication, for example, to external device 12, to server 94, or to another external device. Such an external device may present the indication to a user via, for example, a user interface, such as user interface 86.

In some examples, processing circuitry 50 is configured to identify at least one of a P-wave or one or more data points indicative of a P-wave amongst the identified data points on the cardiac EGM. In some examples, processing circuitry 50 is configured to generate, from the cardiac EGM, a modified cardiac EGM based on the identification of the identified data points. In some examples, the modified cardiac EGM comprises visual indicia for one or more P-waves.

In some examples, processing circuitry 50 is further configured to output the indication for display. In some examples, the one or more arrhythmia detection criteria are indicative of at least one of bradycardia, atrial and/or ventricular tachycardia, atrial and/or ventricular fibrillation, or asystole. In some examples, the training data set comprises EGM data sensed by one or more devices comprising one or more non-atrial sensing electrodes and one or more atrial sensing electrodes. In some examples, IMD 10 further includes communication circuitry 54 communicatively coupled to processing circuitry 50, wherein processing circuitry 50 is configured to control communication circuitry 54 to output the indication to an external device.

In some examples, the non-atrial cardiac EGM data includes synthesized non-atrial cardiac EGM data. In some examples, the synthesized non-atrial cardiac EGM data includes a first non-atrial cardiac EGM data at least one of overlaid, shifted, or error processed with respect to second non-atrial cardiac EGM data. In some examples, the trained machine learning model includes a first trained machine learning model, and the processing circuitry is further configured to apply a second trained machine learning model to the non-atrial cardiac EGM data to determine a manner of synthesizing the synthesized non-atrial cardiac EGM data.

The order and flow of the operation illustrated in FIG. 9 and/or FIG. 10 is one example. In other examples according to this disclosure, the order or flow of the operation of FIG. 9 and/or FIG. 10 may be changed. Further, in some examples, processing circuitry may perform or not perform the operation of FIG. 9 or FIG. 10, or any of the techniques described herein, as directed by a user, e.g., via external device 12 or devices 90. For example, a patient, clinician, or other user may turn on or off functionality for identifying arrhythmia detection remotely (e.g., using Wi-Fi or cellular services) or locally (e.g., using an application provided on a patient's cellular phone or using a medical device programmer).

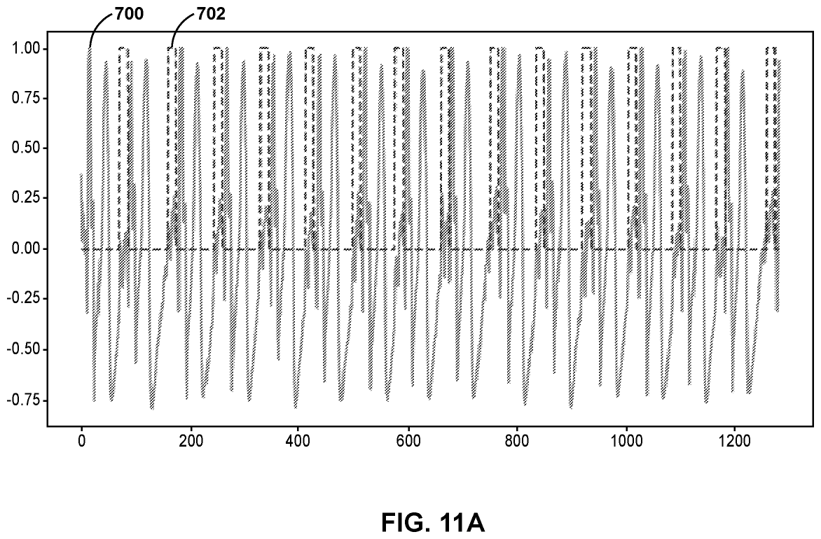

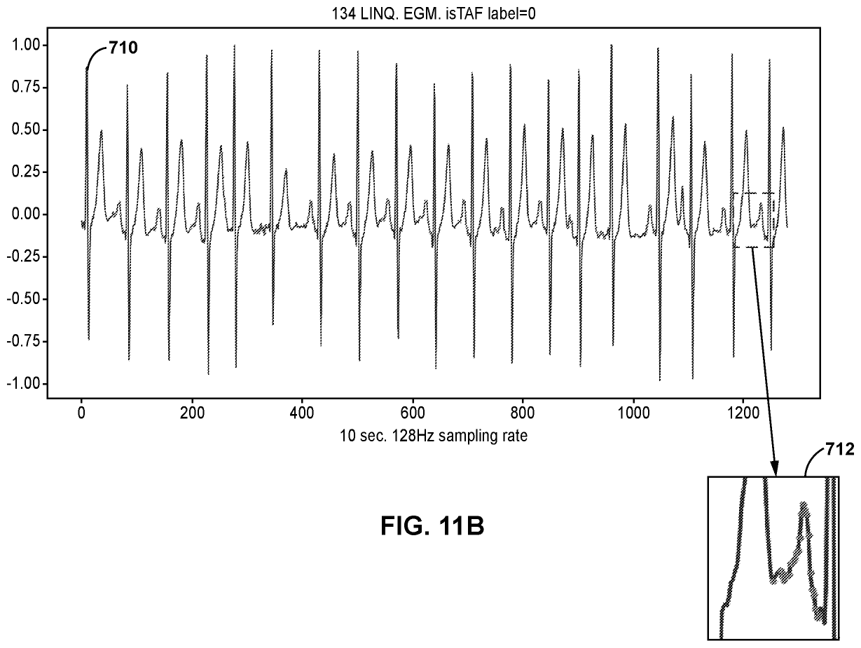

FIG. 11A and FIG. 11B are each an illustration of a cardiac EGM corresponding to an output configuration of a leadless medical device. The respective illustration of FIG. 8A is a synthesized cardiac EGM that results from training a machine learning model from a corpus of intracardiac EGMs from an invasive medical device. The respective illustration of FIG. 8B is a sensed cardiac EGM that results from recording electrical activity of non-atrial tissue and augmenting that sensed cardiac EGM with visual indicators for simulated atrial electrical activity.

FIG. 11A illustrates an example synthesized EGM 700 with ICM-like waveforms derived from ICD waveforms using 0.92*Ventricular lead+0.08 SVC-can lead. It should noted that there are many other possible combinations of leads and coefficients. The dashed lines (e.g., region 702) indicate regions in which the P-waves are located (and atrial sense markers from an atrial lead of the ICD). Machine learning model(s) 97 may be trained in accordance with the present disclosure to predict data points corresponding to locations (e.g., time stamps) of the atrial sense markers and/or locations of ventricular sense markers on the synthesized EGM. The machine learning model can be trained to also predict other statistical measurements corresponding to heart rate variability, such as a mean, standard deviation, entropy of atrial electrical activity (AA) interval, RR intervals (intervals between R-waves), diff(AA) intervals, and/or diff (RR) intervals.

FIG. 11B illustrates a subcutaneous EGM 710 with LINQ waveforms and dots as visual indicators for model-predicted P-wave locations. In the illustration of FIG. 8B, there is a single P-wave in each RR interval. The visual indicators enhance the presentation of the subcutaneous EGM and facilitates AF adjudication. An alternative visual indicator may include enlarged P-waves. A portion 712 of subcutaneous EGM 710 is blown up for easier viewing of the visual indicators.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

This disclosure includes the following non-limiting examples.

Example 1. A medical system comprising: sensing circuitry configured to sense a cardiac electrogram (EGM) of a patient via a plurality of non-atrial electrodes; and processing circuitry configured to: apply a trained machine learning model to the cardiac EGM, the trained machine learning model being previously trained on non-atrial cardiac EGM data and atrial electrical activity labels; based on the application of the trained machine learning model to the cardiac EGM, determine whether at least a portion of the cardiac EGM satisfies one or more arrhythmia detection criteria; and based on the at least a portion of the cardiac EGM satisfying the one or more arrhythmia detection criteria, generate an indication for output, the indication being indicative of the satisfaction of the one or more arrhythmia detection criteria.

Example 2. The medical system of example 1, wherein the processing circuitry is configured to identify at least one of a P-wave or one or more data points indicative of a P-wave amongst the identified data points on the cardiac EGM.

Example 3. The medical system of example 1 or example 2, wherein the processing circuitry is configured to generate, from the cardiac EGM, a modified cardiac EGM based on the identified data points.

Example 4. The medical system of example 3, wherein the modified cardiac EGM comprises visual indicia for one or more P-waves.

Example 5. The medical system of any of examples 1-4, wherein the processing circuitry is further configured to output the indication for display.

Example 6. The medical system of any of examples 1-5, wherein the one or more arrhythmia detection criteria are indicative of at least one of bradycardia, tachycardia, fibrillation, or asystole.

Example 7. The medical system of any of examples 1-6, wherein the training data set comprises EGM data sensed by one or more devices comprising one or more non-atrial sensing electrodes and one or more atrial sensing electrodes.

Example 8. The medical system of any of examples 1-7, further comprising communication circuitry communicatively coupled to the processing circuitry, wherein the processing circuitry is configured to control the communication circuitry to output the indication to an external device.

Example 9. The medical system of any of examples 1-8, wherein the non-atrial cardiac data comprises synthesized non-atrial cardiac EGM data.

Example 10. The medical system of example 9, wherein the synthesized non-atrial cardiac EGM data comprises a first non-atrial cardiac EGM data at least one of overlaid, shifted, or error processed with respect to second non-atrial cardiac EGM data.

Example 11. The medical system of example 9 or example 10, wherein the trained machine learning model comprises a first trained machine learning model, wherein the processing circuitry is further configured to apply a second trained machine learning model to the non-atrial cardiac EGM data to determine a manner of synthesizing the synthesized non-atrial cardiac EGM data.

Example 12. The medical system of any of examples 1-11, wherein the atrial electrical activity labels are determined from atrial cardiac EGM data.

Example 13. The medical system of any of examples 1-12, wherein as part of determining whether the cardiac EGM data satisfies the one or more arrhythmia detection criteria, the processing circuitry is configured to identify data points of the cardiac EGM signal and determine whether the identified data points satisfy the one or more arrhythmia detection criteria.

Example 14. The medical system of any of examples 1-13, wherein the medical system comprises an insertable cardiac monitor, the insertable cardiac monitor comprising: a power source operatively coupled to the processing circuitry; a memory operatively coupled to the processing circuitry and configured to store the machine learning model; a distal electrode operatively coupled to the processing circuitry; a proximal electrode operatively coupled to the processing circuitry; and a hermetically-sealed housing configured for subcutaneous implantation within the patient, wherein at least the power source, memory, and processing circuitry are within the hermetically-sealed case, and wherein the housing has a length, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm.

Example 15. The medical system of any of examples 1-14, wherein the atrial electrical activity labels are based at least in part on atrial electrical activity sensed in intracardiac EGM data, and wherein the trained machine learning model is further previously trained on at least one of non-atrial cardiac EGM data, synthesized non-atrial EGM data, and/or QRS subtracted EGM data.

Example 16. The medical system of any of examples 1-15, wherein the medical system is configured to continuously and without human intervention monitor cardiac EGMs of the patient.

Example 17. A method comprising: applying, using processing circuitry of medical system, a trained machine learning model to a cardiac electrogram (EGM), the trained machine learning model being previously trained on non-atrial cardiac EGM data and atrial electrical activity labels; based on the application of the trained machine learning model to the cardiac EGM, determining whether at least a portion of the cardiac EGM satisfies one or more arrhythmia detection criteria; and based on the at least a portion of the cardiac EGM satisfying the one or more arrhythmia detection criteria, generating an indication for output, the indication being indicative of the satisfaction of the one or more arrhythmia detection criteria.

Example 18. The method of example 17, further comprising identifying at least one of a P-wave or one or more data points indicative of a P-wave amongst the identified data points on the cardiac EGM.

Example 19. The method of example 17 or example 18, further comprising generating, from the cardiac EGM, a modified cardiac EGM based on the identified data points.

Example 20. The method of example 19, wherein the modified cardiac EGM comprises visual indicia for one or more P-waves.

Example 21. The method of any of examples 17-20, further comprising outputting the indication for display.

Example 22. The method of any of examples 17-21, wherein the one or more arrhythmia detection criteria are indicative of at least one of bradycardia, tachycardia, fibrillation, or asystole.

Example 23. The method of any of examples 17-22, wherein the training data set comprises EGM data sensed by one or more devices comprising one or more non-atrial sensing electrodes and one or more atrial sensing electrodes.

Example 24. The method of any of examples 17-23, wherein the non-atrial cardiac EGM data comprises synthesized non-atrial cardiac EGM data.

Example 25. The method of example 24, wherein the synthesized non-atrial cardiac EGM data comprises a first non-atrial cardiac EGM data at least one of overlaid, shifted, or error processed with respect to second non-atrial cardiac EGM data.

Example 26. The method of example 24 or example 25, wherein the trained machine learning model comprises a first trained machine learning model, and wherein the method further comprises applying a second trained machine learning model to the non-atrial cardiac EGM data to determine a manner of synthesizing the synthesized non-atrial cardiac EGM data.

Example 27. The method of any of examples 17-26, wherein the atrial electrical activity labels are determined from atrial cardiac EGM data.

Example 28. The method of any of examples 17-27, wherein determining whether the cardiac EGM data satisfies the one or more arrhythmia detection criteria comprises identifying data points of the cardiac EGM signal and determining whether the identified data points satisfy the one or more arrhythmia detection criteria.

Example 29. The method of any of examples 17-28, wherein the method is performed by a medical system comprising an insertable cardiac monitor, the insertable cardiac monitor comprising: a power source operatively coupled to the processing circuitry; a memory operatively coupled to the processing circuitry and configured to store the machine learning model; a distal electrode operatively coupled to the processing circuitry; a proximal electrode operatively coupled to the processing circuitry; and a hermetically-sealed housing configured for subcutaneous implantation within the patient, wherein at least the power source, memory, and processing circuitry are within the hermetically-sealed case, and wherein the housing has a length, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm.

Example 30. The method of any of examples 17-29, wherein the atrial electrical activity labels are based at least in part on atrial electrical activity sensed in intracardiac EGM data, and wherein the trained machine learning model is further previously trained on at least one of non-atrial cardiac EGM data, synthesized non-atrial EGM data, and/or QRS subtracted EGM data.

Example 31. The method of any of examples 17-30, further comprising continuously and without human intervention monitoring cardiac EGMs of the patient.

Example 32. A non-transitory computer-readable storage medium comprising program instructions that, when executed by processing circuitry of a medical system, cause the processing circuitry to: apply a trained machine learning model to the cardiac EGM, wherein a training data set of the trained machine learning model comprises non-atrial cardiac EGM data and atrial electrical activity labels, wherein the atrial electrical activity labels are determined from atrial cardiac EGM data corresponding to the non-atrial cardiac EGM data; based on the application of the trained machine learning model to the cardiac EGM, identify data points of the cardiac EGM indicative of atrial electrical activity; based on the identification of the data points, determine whether the cardiac EGM satisfies one or more arrhythmia detection criteria; and based on the cardiac EGM satisfying the one or more arrhythmia detection criteria, generate an indication for output, the indication being indicative of the satisfaction of the one or more arrhythmia detection criteria.

1. A medical system comprising:
sensing circuitry configured to sense a cardiac electrogram (EGM) of a patient via a plurality of non-atrial electrodes; and
processing circuitry configured to:
apply a trained machine learning model to the cardiac EGM, the trained machine learning model being previously trained on non-atrial cardiac EGM data and atrial electrical activity labels;
based on the application of the trained machine learning model to the cardiac EGM, determine whether at least a portion of the cardiac EGM satisfies one or more arrhythmia detection criteria; and
based on the at least a portion of the cardiac EGM satisfying the one or more arrhythmia detection criteria, generate an indication for output, the indication being indicative of the satisfaction of the one or more arrhythmia detection criteria.

2. The medical system of claim 1, wherein the processing circuitry is configured to identify at least one of a P-wave or one or more data points indicative of a P-wave amongst identified data points on the cardiac EGM.

3. The medical system of claim 1, wherein the processing circuitry is configured to generate, from the cardiac EGM, a modified cardiac EGM based on the identified data points.

4. The medical system of claim 3, wherein the modified cardiac EGM comprises visual indicia for one or more P-waves.

5. The medical system of claim 1, wherein the processing circuitry is further configured to output the indication for display.

6. The medical system of claim 1, wherein the one or more arrhythmia detection criteria are indicative of at least one of bradycardia, tachycardia, fibrillation, or asystole.

7. The medical system of claim 1, wherein the a training data set comprises EGM data sensed by one or more devices comprising one or more non-atrial sensing electrodes and one or more atrial sensing electrodes.

8. The medical system of claim 1, further comprising communication circuitry communicatively coupled to the processing circuitry, wherein the processing circuitry is configured to control the communication circuitry to output the indication to an external device.

9. The medical system of claim 1, wherein the non-atrial cardiac EMG data comprises synthesized non-atrial cardiac EGM data.

10. The medical system of claim 9, wherein the synthesized non-atrial cardiac EGM data comprises a first non-atrial cardiac EGM data at least one of overlaid, shifted, or error processed with respect to second non-atrial cardiac EGM data.

11. The medical system of claim 9 wherein the trained machine learning model comprises a first trained machine learning model, wherein the processing circuitry is further configured to apply a second trained machine learning model to the non-atrial cardiac EGM data to determine a manner of synthesizing the synthesized non-atrial cardiac EGM data.

12. The medical system of claim 1, wherein the atrial electrical activity labels are determined from atrial cardiac EGM data.

13. The medical system of claim 1, wherein as part of determining whether the cardiac EGM data satisfies the one or more arrhythmia detection criteria, the processing circuitry is configured to identify data points of the cardiac EGM and determine whether identified data points satisfy the one or more arrhythmia detection criteria.

14. The medical system of claim 1, wherein the medical system comprises an insertable cardiac monitor, the insertable cardiac monitor comprising:
a power source operatively coupled to the processing circuitry;
a memory operatively coupled to the processing circuitry and configured to store a machine learning model;
a distal electrode operatively coupled to the processing circuitry;
a proximal electrode operatively coupled to the processing circuitry; and
a hermetically-sealed housing configured for subcutaneous implantation within the patient, wherein at least the power source, memory, and processing circuitry are within the hermetically-sealed housing, and
wherein the hermetically-sealed housing has a length, a width, and a depth,
wherein the length is greater than the width and the width is greater than the depth,
wherein the length is within a range from 5 millimeters (mm) to 60 mm,
wherein the width is within a range from 5 mm to 15 mm, and
wherein the depth is within a range from 5 mm to 15 mm.

15. The medical system of claim 1, wherein the atrial electrical activity labels are based at least in part on atrial electrical activity sensed in intracardiac EGM data, and wherein the trained machine learning model is further previously trained on at least one of non-atrial cardiac EGM data, synthesized non-atrial EGM data, and/or QRS subtracted EGM data.

16. The medical system of claim 1, wherein the medical system is configured to continuously and without human intervention monitor cardiac EGMs of the patient.

17. A method comprising:
applying, using processing circuitry of medical system, a trained machine learning model to a cardiac electrogram (EGM), the trained machine learning model being previously trained on non-atrial cardiac EGM data and atrial electrical activity labels;
based on the application of the trained machine learning model to the cardiac EGM, determining whether at least a portion of the cardiac EGM satisfies one or more arrhythmia detection criteria; and
based on the at least a portion of the cardiac EGM satisfying the one or more arrhythmia detection criteria, generating an indication for output, the indication being indicative of the satisfaction of the one or more arrhythmia detection criteria.

18. : The method of claim 17, further comprising identifying at least one of a P-wave or one or more data points indicative of a P-wave amongst the identified data points on the cardiac EGM.

19. : The method of claim 17, further comprising generating, from the cardiac EGM, a modified cardiac EGM based on the identified data points, wherein the modified cardiac EGM comprises visual indicia for one or more P-waves.

20. : A non-transitory computer-readable storage medium comprising program instructions that, when executed by processing circuitry of a medical system, cause the processing circuitry to:
apply a trained machine learning model to a cardiac electrogram (EGM), wherein a training data set of the trained machine learning model comprises non-atrial cardiac EGM data and atrial electrical activity labels, wherein the atrial electrical activity labels are determined from atrial cardiac EGM data corresponding to the non-atrial cardiac EGM data;
based on the application of the trained machine learning model to the cardiac EGM, identify data points of the cardiac EGM indicative of atrial electrical activity;
based on the identification of the data points, determine whether the cardiac EGM satisfies one or more arrhythmia detection criteria; and
based on the cardiac EGM satisfying the one or more arrhythmia detection criteria, generate an indication for output, the indication being indicative of the satisfaction of the one or more arrhythmia detection criteria.
